(19) United States
(12) Patent Application Publication
(10) Pub. No.: US 2025/0323532 A1
(43) Pub. Date: Oct. 16, 2025

(54) CHARGING INTERFACE COMMUNICATION

(71) Applicant: Oura Health Oy, Oulu (FI)

(72) Inventors: Kalle Juho Säippä, Oulu (FI); Miika Petteri Kanste, Oulu (FI)

(21) Appl. No.: 18/636,043

(22) Filed: Apr. 15, 2024

(51) Int. Cl. H02J 50/80; G06F 1/16; H02J 7/00; H02J 50/10

(52) U.S. Cl. CPC H02J 50/80; G06F 1/163; H02J 7/00034; H02J 7/00306; H02J 7/0071; H02J 7/007182; H02J 50/10

(57) ABSTRACT

Methods, systems, and devices for a wearable device are described. A wearable device (e.g., ring, watch) may charge via interacting with a charging device (e.g., charger). The wearable device may receive, via inductive charging components, a sequence of incrementally decreasing power output levels from the charging device, where the power output levels correspond to detected voltage levels at the wearable device. The wearable device may activate charging-based communication circuitry within the wearable device upon detecting a first power output level of the sequence of incrementally decreasing power output levels that falls below an overload protection threshold, and output a first message to the charging device. The wearable device may receive, from the charging device, a second message in response to the first message.

### FIELD OF TECHNOLOGY

The following relates to wearable devices and data processing, including charging interface communication.

### BACKGROUND

Some wearable devices may be configured to collect data from users. Some wearable devices may be designed to include one or more elements that may facilitate charging via a charging device (e.g., a charger).

### BRIEF DESCRIPTION OF THE DRAWINGS

FIG. 1 illustrates an example of a system that supports charging interface communication in accordance with aspects of the present disclosure.

FIG. 2 illustrates an example of a system that supports charging interface communication in accordance with aspects of the present disclosure.

FIG. 3 shows an example of a charging diagram that supports charging interface communication in accordance with aspects of the present disclosure.

FIG. 4 shows an example of a charging flow diagram that supports charging interface communication in accordance with aspects of the present disclosure.

FIG. 5 shows an example of a charging flow diagram that supports charging interface communication in accordance with aspects of the present disclosure.

FIG. 6 shows an example of a process flow diagram that supports charging interface communication in accordance with aspects of the present disclosure.

FIG. 7 shows a block diagram of an apparatus that supports charging interface communication in accordance with aspects of the present disclosure.

FIG. 8 shows a block diagram of a wearable device manager that supports charging interface communication in accordance with aspects of the present disclosure.

FIG. 9 shows a diagram of a system including a device that supports charging interface communication in accordance with aspects of the present disclosure.

FIG. 10 shows a block diagram of an apparatus that supports charging interface communication in accordance with aspects of the present disclosure.

FIG. 11 shows a block diagram of a wearable application that supports charging interface communication in accordance with aspects of the present disclosure.

FIG. 12 shows a diagram of a system including a device that supports charging interface communication in accordance with aspects of the present disclosure.

FIGS. 13 through 16 show flowcharts illustrating methods that support charging interface communication in accordance with aspects of the present disclosure.

### DETAILED DESCRIPTION

A wearable device, such as a ring or watch, may be configured to measure data, such as biometric data, of a user and report the data to the user. The wearable device may be charged via a charging device (e.g., charger), and may be charged using contact-based (e.g., galvanic) charging. However, abrasions, chemicals, skin particles, and dirt may all negatively impact the efficacy of galvanic charging. Infrared signals may be used in addition to galvanic charging to transfer data, such as messages, between the wearable device and the charging device. However, infrared communications between the wearable device and the charging device may be inadvertently detected by nearby wearables, which may result in confusion and inefficacy in the communications. Additionally, in order for infrared communication between the charging device and wearable device to work, the materials or colors of the charging device or wearable device may be limited. The drawbacks of galvanic charging and the lack of flexibility and additional components required for infrared communication present additional manufacturing costs and complexity. Improving the communication method between the charging device and wearable device would be advantageous for production costs, design flexibility, and communication reliability.

Techniques described herein provide for a charging interface communication method that implements inductive charging. Inductive charging, or wireless charging, may not require the charging device to have an infrared sensor, nor a specific material color or finish, and is not as negatively impacted by the physical state of the wearable device (such as dirt, chemicals, abrasions, etc.) as galvanic charging. The wearable device may communicate by modulating a power draw, which is detected by the charger, and the charger communicates by modulating the power output, which is detected by the wearable device. To establish a communication link, the charging device output power may match the wearable device input power that the wearable device needs to be fully charged. This steady state charging power is variable, as the wearable device may be at varying states of charge depending on when the wearable is placed on the charger. The charging device outputs power and listens for a message from the wearable device. If a message is not received, the charging device reduces the output power. The charging device continues listening and reducing power until receiving a message. In general, the wearable device communicates to the charger by modulating the load current expressed at the charger device. At the same time, the wearable device receives the power, and waits until the power has been reduced below a threshold (e.g., an overload threshold), and then transmits a message to the charger device by drawing power. In general, the charger device communicates to the wearable device by modulating the charging voltage expressed at the wearable device. For example, the power draw may be interpreted as one or more bits by the charging device, indicating a first message, such as a link establishment message. The charging device may then modulate the output power, which may be interpreted by the wearable device as one or more bits, indicating a second message, such as a link confirmation message.

Aspects of the disclosure are initially described in the context of systems supporting physiological data collection from users via wearable devices. Aspects of the disclosure are further illustrated by and described with reference to charging diagrams, charging flow diagrams, and process flow diagrams. Aspects of the disclosure are further illustrated by and described with reference to apparatus diagrams, system diagrams, and flowcharts that relate to charging interface communication.

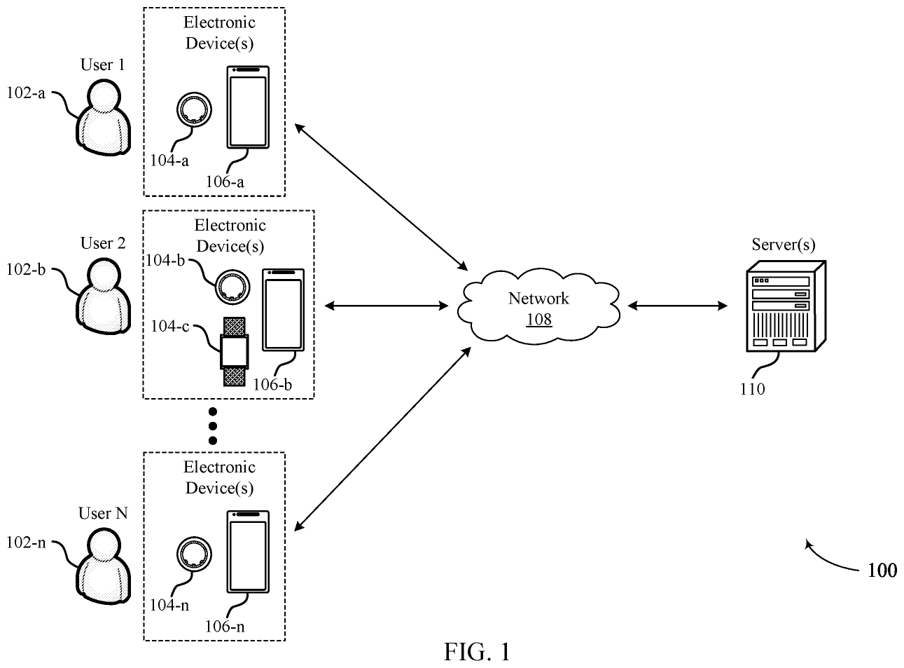

FIG. 1 illustrates an example of a system 100 that supports charging interface communication in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols. Moreover, in some cases, the wearable device 104 and the user device 106 may be included within (or make up) the same device. For example, in some cases, the wearable device 104 may be configured to execute an application associated with the wearable device 104, and may be configured to display data via a GUI.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for charging interface communication. The charging interface communication method described herein may implement messages transmitted via inductive charging. The wearable device 104 may communicate by modulating a power draw, which is detected by the charger, and the charger communicates by modulating the power output, which is detected by the wearable device 104.

To establish a link, the charging device detects the proximity of the wearable device 104, and outputs a starting, or first, maximum power level. The charging device may then begin a timer, and wait for a message from the wearable device 104. If a message is not received prior to expiration of the timer, the charging device reduces the output power. The charging device continues listening and reducing power until receiving a message from the wearable device 104. At the same time, the wearable device 104 receives the power, and waits until the power has been reduced below a threshold, and then transmits a message by modulating a power draw. The threshold may be a power level, or charging level, of the wearable device 104.

The modulation in power draw may be interpreted as one or more bits by the charging device, indicating one or more messages, such as a link establishment message. The charging device may then modulate the output power, which may be interpreted by the wearable device as one or more bits, indicating one or more messages, such as a link confirmation message. After link establishment, the wearable device 104 and charging device may communicate messages by modulating power output and power draw. For example, the wearable device 104 may indicate to the charging device to increase or decrease the power output.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

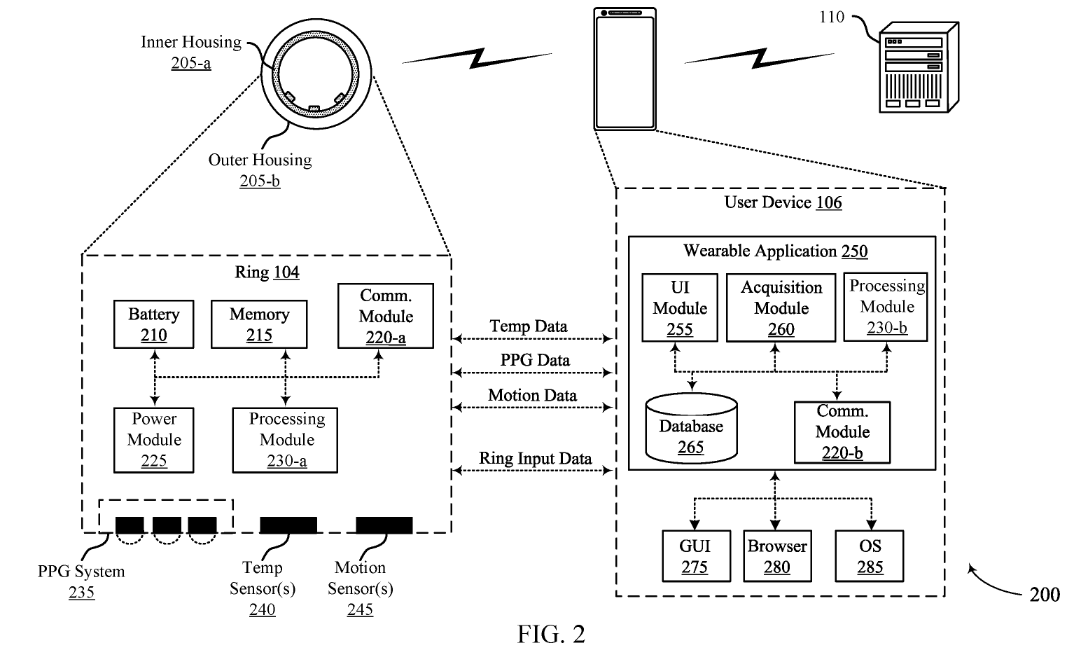

FIG. 2 illustrates an example of a system 200 that supports charging interface communication in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time.

For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

In some cases, the wearable device 104 and the user device 106 may be included within (or make up) the same device. For example, in some cases, the wearable device 104 may be configured to execute the wearable application 250, and may be configured to display data via the GUI 275.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 may support techniques for charging interface communication. The charging interface communication method described herein may include messages transmitted via inductive charging modulation. The wearable device 104 may communicate by modulating a power draw, which is detected by the charger and interpreted as one or more bits, and the charging device may communicate by modulating the power output, which is detected by the wearable device 104 and interpreted as one or more bits.

To establish a link, the charging device detects the proximity of the wearable device 104, and outputs a starting, or first, maximum power level. The charging device may then begin a timer, and wait for a message from the wearable device 104. If a message is not received prior to expiration of the timer, the charging device reduces the power and restarts the timer. The charging device continues waiting for a message and reducing power until receiving a message. At the same time, the wearable device 104 receives and detects the power, and waits until the power has been reduced below a threshold, and then transmits a message by modulating a power draw. The threshold may be a power level, or charging level, of the wearable device 104. The message indicated by the wearable device 104 when the power level satisfies the threshold may be a link establishment message, or some other message. The charging device may receive the link establishment message and send a link confirmation message to establish the charging link. Although examples herein describe message transfer related to link establishment (e.g., link establishment message and corresponding link confirmation message), it is to be understood that the techniques described herein may apply to other types of messaging purposes in addition to or in alternative to link establishment. Once the link has been established, the charging device and wearable device 104 may communicate messages throughout the charging process (e.g., link maintenance messages, charging status messages, messages related to physiological or environmental data collected by the wearable device, messages related to physiological or environmental data collected by the charger device, etc.).

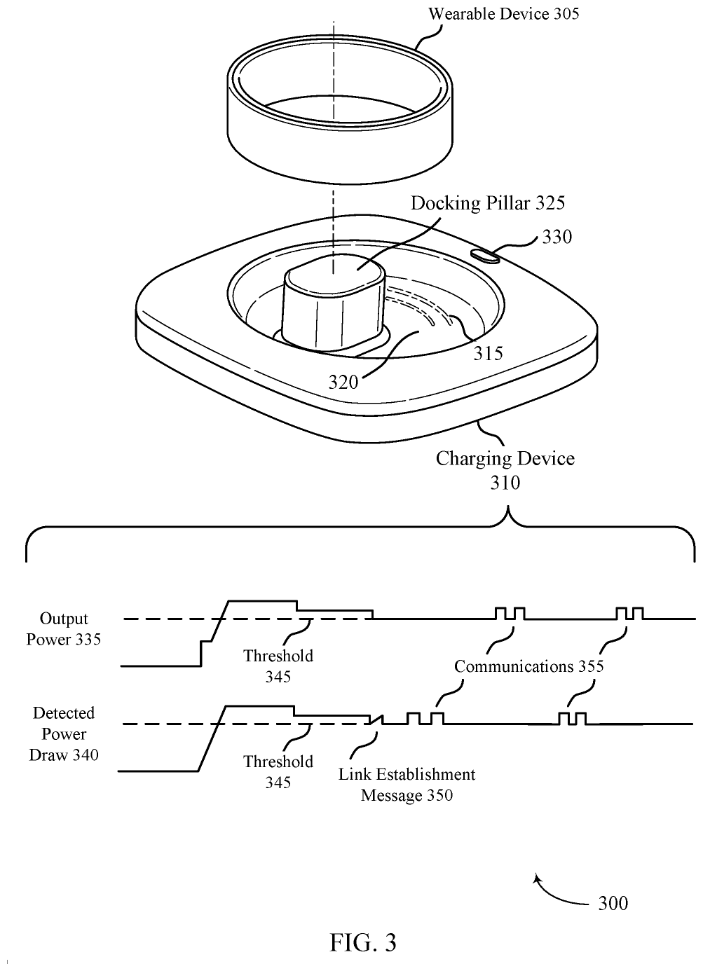

FIG. 3 shows an example of a charging diagram 300 that supports charging interface communication in accordance with aspects of the present disclosure. The charging diagram 300 may illustrate a wearable device 305 and a charging device 310. The wearable device 305 may charge via the charging device 310 according to the charging interface communication methods described herein. In some implementations, the charging diagram 300 may implement, or be implemented by, aspects of the system 100, the system 200, or a combination thereof. The wearable device 305 (e.g., device, wearable ring device, ring, wearable wrist-worn device) may be an example of the wearable device 104 (e.g., the ring 104) as described with reference to FIGS. 1-2.

The charging device 310 (e.g., charger, charging station) may include a receptacle portion 320 that is configured to receive and interface with wearable devices, such as the wearable device 305. Although shown as a recessed portion, the receptacle portion 320 may be a flat surface (e.g., disc-shaped) or a raised surface. In some examples, the receptacle portion 320 may include one or more retention sub-portions 315, also referred to as a magnetic sub-portion, that is magnetic. The one or more retention sub-portions 315 may apply an attractive force to a wearable device 305 so that the wearable device 305 is more forcefully fixed in the charging position, which may prevent the wearable device 305 from being inadvertently jarred loose. The retention sub-portion 315 may be a single strip of magnetic material or may be one or multiple retention sub-portions.

The charging device 310 may also include a docking pillar 325 that is configured to fit within the inner circumferential surface of the wearable device 305. In some examples, the docking pillar 325 may be configured to fit within the inner circumferential surfaces of differently sized wearable device 305. The docking pillar 325 may be any shape, such as circular or semi-circular, and may not directly contact the wearable device 305. In some examples, the charging device 310 may not include a docking pillar 325. The charging device may also include an indicator 330, which may indicate charging status, such as by changing color. For example, the indicator 330 may be green when charging is complete, red while charging, and flash red if the wearable device 305 is not properly connected to the charging device 310 or to indicate some other error. The indicator 330 may be another sensor, or another element related to charging. In some examples, the charging device 310 may not include the indicator 330.

The wearable device 305 may include inductive charging components and charging-based communication circuitry that may be activated or otherwise implemented to charge and communicate with the charging device 310 while charging. The charging device 310 may include inductive charging components and charging-based communication circuitry that may be employed to charge the wearable device 305 and communicate with the wearable device 305. Communications may include link establishment, link status, charging status, charging information, physiological data transfer, environmental data transfer, and other communications (e.g., charging fault or other investigation and remedy-related communication between the charging device 310 and the wearable device 305, or other negotiations) between the devices that may be advantageous.

The charging device 310 and the wearable device 305 may communicate various messages by modulating power output and power draw. The charging device 310 may output power 335 and detect when power is drawn by the wearable device 305 (e.g., detected power draw 340). The output power 335 levels may correspond to detected voltage levels at the wearable device 305. That is, if the charging device 310 outputs more power, the wearable device 305 may detect increased voltage levels. The wearable device 305 may draw additional power in quick bursts and/or according to a predefined pattern to communicate messages. The charging device 310 may detect the additional power drawn from the wearable device as a detected power draw 340, and may interpret the modulations in power draws as one or more messages (e.g., as a set of bits) from the wearable device 305.

To communicate messages, the charging device 310 and the wearable device 305 may perform an initial connection procedure, or link establishment procedure. The charging device 310 may detect that the wearable device 305 is present (e.g., by detecting an initial power draw). The charging device may output power 335 in incrementally decreasing levels until reaching the threshold 345. The wearable device 305 may receive the output power 335 from the charging device 310, and wait until the output power 335 reaches a level that satisfies the threshold 345. Once the output power 335 level reaches the threshold 345, the wearable device 305 may output a link establishment message 350 (or some other message) by modulating a power draw. The charging device 310 may detect the modulations in power draw, resulting in an increased detected power draw 340.

After establishing the link, the charging device 310 and the wearable device 305 may communicate additional messages, or communications 355. The charging device may increase output power 335 to output communications 355, and may detect power drawn from the wearable device 305 to output communications 355. The power draw and output may be interpreted as a series of bits, and further interpreted as a message. The link establishment message and other messages may be functionally the same. That is, while referred to herein as ‘link establishment message’ and ‘additional message’ to clarifying timing and purpose, the method of communicating messages may be applied to any type of message.

The threshold 345 may be an overload protection threshold, which may be based on the charge level of the wearable device 305 or some other static or dynamically defined charging level. For example, the wearable device 305 is nearly fully charged, the wearable device 305 may have a lower threshold 345. If the wearable device 305 has no charge, the wearable device 305 may indicate the link establishment message 350 after the first output power 335 level from the charging device 310.

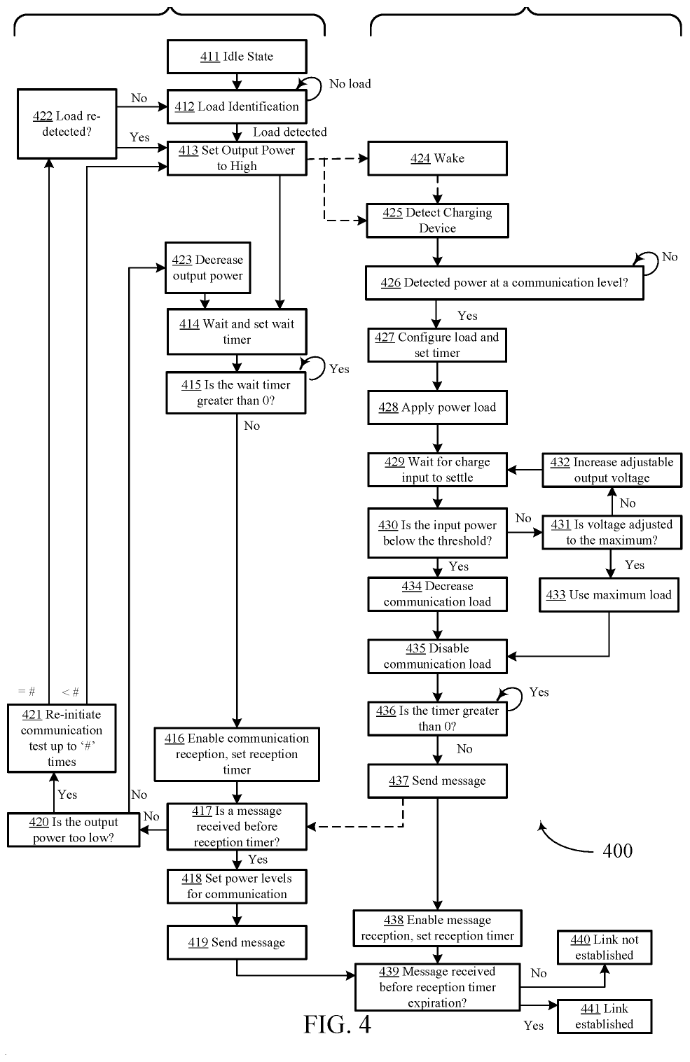

FIG. 4 shows an example of a charging flow diagram 400 that supports charging interface communication in accordance with aspects of the present disclosure. The charging flow diagram 400 may describe a process for communicating messages between a wearable device 405 and a charging device 410. In one example, charging flow diagram 400 describes a communication process for establishing and maintaining a communication link between a wearable device 405 and a charging device 410. However, the charging flow diagram 400 may also be applied to the communication of generic messages in addition to or in alternative to link establishment messages. In some implementations, the charging flow diagram 400 may implement, or be implemented by, aspects of the system 100, the system 200, the charging diagram 300, or a combination thereof. The wearable device 405 (e.g., device, wearable ring device, ring) may be an example of the wearable device 104 (e.g., the ring 104) as described with reference to FIGS. 1-2. The charging device 410 may be an example of the charging device as described with reference to FIG. 3.

The charging device 410 may be in an idle state prior to detecting the wearable device 405. For example, at 411, the idle state may include starting the microcontroller unit (MCU) of the charging device 410, activating other circuitry, completing self-diagnostic tests, etc. At 412, the charging device 410 may perform load identification. Performing load identification may include measuring a power draw (e.g., from a connected wearable device 405). For example, a parameter (e.g., Output) may be activated (e.g., set as ‘ON’), which may trigger various other parameters. Other parameters may include VBOOST being set to 50%, COIL_SWITCH set to oscillate between ON and OFF (e.g., 100 ms ON, 1000 ms OFF), and the Monitor Booster Input Current to increase. If no load is detected, the charging device 410 may, at 412, repeat the load identification process at a configured periodicity. If a load is detected, the charging device 410 may, at 413, set the output power to a maximum level, or ‘high’.

At 413, the charging device 410 may set the output power to ‘high’ in response to detecting the load, or detecting the wearable device 405. In some examples, the wearable device 405 may also detect the charging device 410. The highest output power, or maximum output power, may be preconfigured or otherwise determined. Setting the output power may include changing the parameter VBOOST_PWM to 50%.

Following setting, and transmitting, the high output power, the charging device 410 may, at 414, wait and set a wait timer. The charging device 410 may wait for the wearable device 405 to complete an adjustment. The charging device 410 may also mute any data links and set a wait timer. The wait timer may be a timer (e.g., BLANK_TIMER=50 ms) that defines the waiting time. At 415, the charging device 410 may check if the wait timer is greater than 0. If the wait timer is greater than zero, indicating the wait timer has not expired, the charging device 410 may continue to wait. If the wait timer is not greater than 0, indicating that the wait timer has expired, the charging device may continue to step 416.

At 416, upon expiration of the timer, the charging device 410 may enable communication reception and begin a reception timer. The reception timer may be CHARGER_RX_TIMEOUT, and may define a time (e.g., 50 ms) the charging device 410 waits to receive a message from the wearable device 405. The message may be a first message, such as a link establishment message. The message may be detected at the charging device 410 based on detecting a modulation (e.g., an increased) in power draw by the wearable device 405.

At 417, the charging device 410 may determine if a link establishment message (or some other message) was received before the expiration of the reception timer. If the charging device 410 detected a power draw modulation prior to the expiration of the reception timer, the charging device 410 may determine that a message was received, and continue to step 418. If not, the charging device 410 may proceed to evaluate and change the power output starting at 420.

If the charging device 410 received the link establishment message, the charging device 410 may, at 418, set power levels (e.g., voltage levels) for communication. Such levels may be representative of power output, and may include various VBOOST voltage level parameters (e.g., VBOOST−NORMAL−PWM=VBOOST_PWM, VBOOST−COMM−PWM=VBOOST_PWM3). After setting the power, or voltage, levels for communication, the charging device 410 may output a message, such as link confirmation message. At 419, the charging device 410 may send, or otherwise output, a link confirmation message. The message may be communicated by modulating power output, such as quickly increasing the power output in short bursts or according to some other predefined pattern. The power changes may be interpreted by the wearable device 405 as bits and converted to a message. Steps 417-419 may be part of successful link establishment with the wearable device 405, and may enable the communication of future messages over the established link.

If, at 417, the charging device 410 has not received a message (e.g., a link establishment message) prior to the expiration of the reception timer, the charging device 410 may complete a series of steps to modulate the power and transmit incrementally decreasing output power levels until receiving a message from the wearable device 405.

At 420, the charging device 410 may determine if the output power (e.g., voltage level) was lower than a threshold. If the output power is too low, or lower than the threshold, it may be possible that there is an issue with communication with the wearable device 405. For example, if the charging device 410 has reduced the output power multiple times and has not received a message from the wearable device 405, there may be an issue that is unrelated to power output. The wearable device 405 may not be in close enough proximity, or there may be another problem preventing link establishment.

Thus, if at 420 the output power is too low, the charging device 410 may re-initiate the communication test. The charging device 410 may re-initiate communication by repeating step 413, setting the output power to high, and continuing through the process again. However, the charging device 410 may only attempt to re-initiate communication for a specified number of times (e.g., #). For example, the charging device 410 may attempt to re-initiate communication if the attempts (e.g., RETRY_COUNTER) are less than a set maximum, such as up to four times. At the fourth time, the charging device 410 may proceed to step 422.

At 422, the charging device 410 may determine if the load has been re-detected, or if the wearable device 405 has been re-attached or otherwise reconnected. If so, the charging device 410 may attempt again to initiate communication, by setting the output power to high at 413. If there is no wearable device re-attachment detected, the charging device 410 may return to 412 load identification until detecting a load.

Returning to step 420, if the output power does not satisfy a threshold, or is not too low, the charging device 410 may decrease the output power (e.g., voltage) at 423. After decreasing the output power, the charging device 410 may (at 414) wait for a response from the wearable device 405 until the expiration of the wait timer. The decrease of output power may be an incremental step, such as a predefined step. If the charging device 410 does not receive a response when proceeding through steps 414-417, the charging device 410 continues to decrease the output power until the output power is too low (at 420).

In summary, the charging device 410 identifies (at 412) the wearable device 405, (at 413) outputs a high power, and then proceeds in a loop (steps 414-417, 420, 423) of waiting for a response from the wearable device 405, decreasing the output power, and waiting again. The charging device 410 incrementally decreases the output power until (at 417) the charging device 410 receives a message or until (at 420) the output power is too low. The charging device 410 then attempts to detect the wearable device 405 before again initiating communications.

The wearable device 405 has a process flow that interacts with the process flow of the charging device 410. The wearable device 405 receives the output power, determines if the output power is greater than a threshold, and then either communicates a link establishment message (or other message) or waits for the charging device 410 to decrease the power output.

At 424 and 425, the wearable device 405 may wake and detect the charging device 410. At 424, for example, the wearable device 405 may have no charge and must first wake, or boot, to begin interacting with the charging device 410. In some examples, the wearable device 405 may already be awake and able to detect the charging device 410. At 425, the wearable device 405 may detect the charging device 410, and if the charger output power (e.g., power received by the wearable device 405) is within a range, the wearable device 405 may automatically begin establishing a link with the charging device 410. The range may be a power range defined by a manufacturer or another standard.

After detecting the charging device 410, the wearable device 405 may at 426 determine if the power from the charging device 410 received and detected is at a level suitable for communication. For example, the wearable device 405 may compare the received power level (e.g., input power) to a communication power threshold. The power detected may be VCHARGER and the threshold may be Vz. If the power level is not at a level suitable for communication, or is higher than the threshold, the wearable device 405 may continue to wait.

If the detected power is at a suitable level for communication, or lower than the threshold, the wearable device 405 may continue to 427 and configure a voltage transmit load (e.g., power or voltage load), or a communication power load. The communication power load may be a voltage, such as 1 volt. The load may be represented by a parameter (e.g., V_TX_LOAD for a PMIC LDO L2OUT adjustable output). The wearable device 405 may also mute any data links and set a timer (e.g., BLANK_TIMER=50 ms).

At 428, the wearable device 405 may apply the load, or voltage, to a load resistor (e.g., 330R) which may enable an output parameter (e.g., L2OUT Output). The wearable device 405 may at 429 wait for the charge input (e.g., VCHARGER) to settle. The wait period may be defined (e.g., 2 ms).

After waiting for the charge input to settle, the wearable device 405 may determine at 430 if the input power is below a threshold. For example, the wearable device 405 may compare the detected input power (e.g., charge input, detected voltage, VCHARGER), to a threshold. The threshold may be a maximum input charge value the wearable device 405 is capable of receiving. The threshold may depend on the charge level of the wearable device 405. If the wearable device 405 is at a higher charge level, the threshold may be lower, and vice versa. If the input power is below the threshold, the wearable device 405 may proceed to 434. If the input power is not below the threshold, or is too high, the wearable device 405 may proceed to 431 for further diagnostics.

At 431, if the input power is not below the threshold, and is too high, the wearable device 405 may determine if the voltage is adjusted to the maximum (e.g., V_TX_LOAD=4V). If the load is not adjusted to the maximum, the wearable device 405 may at 432 increase the adjustable output voltage (e.g., V_TX_COMM=V_TX_LOAD+0.3 V) and then at 429 wait again for the charge input to settle. If at 431 the voltage is adjusted to the maximum, the wearable device 405 may at 433 use the maximum load for communication (V_TX_COMM=V_TX_LOAD) and proceed to 435 and disable the communication load.

If at 430 the input power is below the threshold, the wearable device 405 may proceed to 434. At 434 the wearable device 405 may decrease a communication load. Decreasing the communication load may implement a safe margin for the detected input power (e.g., V_TX_COMM=V_TX_LOAD-0.3 V).

At 435, the wearable device 405 may disable the communication load (e.g., disable L2OUT output). At 436, the wearable device 405 may check if the timer set at 427 is greater than zero. If the timer (e.g., BLANK_TIMER) is greater than 0, or has not expired, the wearable device 405 may wait until the timer expires.

If the timer is not greater than 0, or has expired, the wearable device 405 may at 437 enable communications and send a message. The message may be communicated by quickly drawing power in one or more short bursts. The message may be a link establishment message. The wearable device 405 may indicate the message using a predetermined communication level (e.g., V_TX_COMM level). Enabling communications may include activating circuitry within the wearable device 405 related to communications with the charging device 410.

At 438, after indicating the message, the wearable device 405 may enable message reception and set a reception timer (e.g., RING_RX_TIMEOUT=50 ms). At 439, the wearable device 405 may determine if a message from the charging device 410 is received prior to expiration of the reception timer. If the message is not received prior to the expiration of the reception timer, the wearable device 405 determines that at 440 the link is not established. If the wearable device 405 does receive a message from the charging device 410, the wearable device 405 may determine at 441 that the link is established.

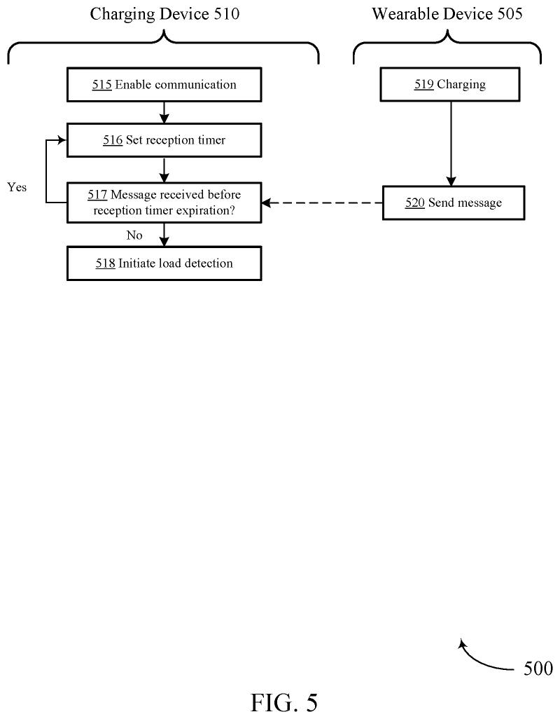

FIG. 5 shows an example of a charging flow diagram 500 that supports charging interface communication in accordance with aspects of the present disclosure. The charging flow diagram 500 describes communications between a charging device 510 and a wearable device 505 after initial link establishment. In some implementations, the charging flow diagram 500 may implement, or be implemented by, aspects of the system 100, the system 200, the charging diagram 300, the charging flow diagram 400, or a combination thereof. The wearable device 505 (e.g., device, wearable ring device, wearable wrist device, ring) may be an example of the wearable device 104 (e.g., the ring 104) as described with reference to FIGS. 1-2. The charging device 510 (e.g., charger) may be an example of the charging device 310 as described with reference to FIG. 3.

After establishing a link, the charging device 510 and the wearable device 505 may communicate one or more additional messages. At 515, the charging device 510 may enable communications, such as receiving messages from the wearable device 505 (e.g., RX ENABLE=TRUE). The charging device 510 may then, at 516, set a reception timer (e.g., CHARGER_RX_TIMEROUT=5000 ms). The wearable device 505 may be in a charging state, at 519. The wearable device 505 may periodically send messages at 520 to the charging device 510.

At 517, the charging device 510 may determine if a message from the wearable device 505 was received prior to the expiration of the reception timer (e.g., CHARGER_RX_TIMEROUT>0?). If the charging device 510 has not received a message and the timer has reached zero, the charging device 510 may move to step 518 and initiate load detection, such as described in FIG. 4. If the charging device 510 does receive a message, the charging device 510 may reset the reception timer and wait for another message.

The message may be indicated via modulations (e.g., rapid draws) of power from the wearable device 505. Messages may include indicating to the charging device 510 to keep the power level the same, increase the power level, or decrease the power level. In some examples, the wearable device 505 may be set to transmit a message periodically, such as every second.

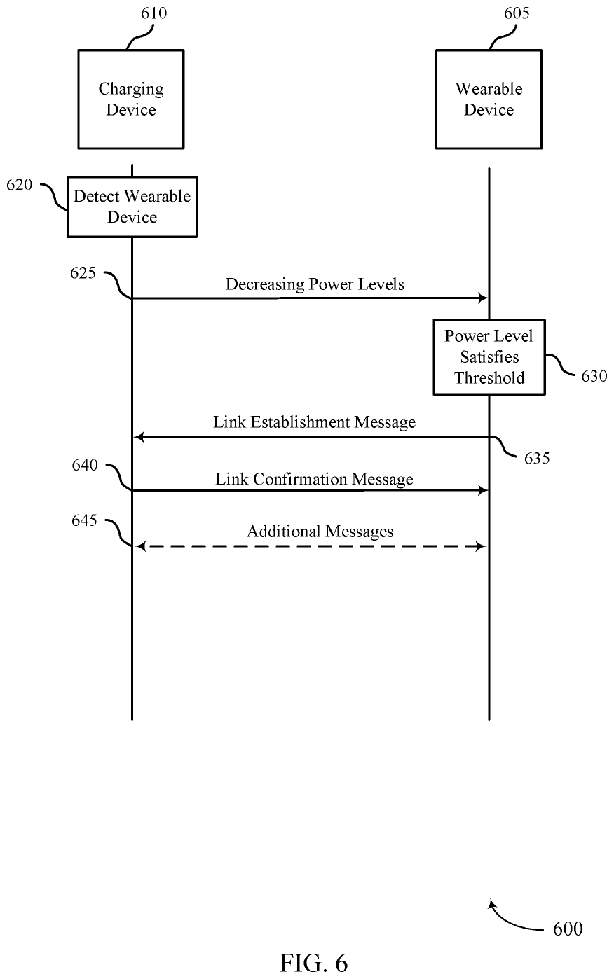

FIG. 6 shows an example of a process flow diagram 600 that supports charging interface communication in accordance with aspects of the present disclosure. The process flow diagram 600 illustrates and describes communicating messages between a charging device 610 and a wearable device 605. In some examples, the communication process may involve establishing a communication link between a charging device 610 and a wearable device 605. In some implementations, the process flow diagram 600 may implement, or be implemented by, aspects of the system 100, the system 200, the charging diagram 300, the charging flow diagram 400, the charging flow diagram 500, or a combination thereof. The wearable device 605 (e.g., device, wearable ring device, ring) may be an example of the wearable device 104 (e.g., the ring 104) as described with reference to FIGS. 1-2. The charging device 610 may be an example of the charging device 305 as described with reference to FIG. 3.

In the process flow diagram 600, the operations between the wearable device 605 and the charging device 610 may be performed in different orders or at different times. Some operations may also be left out of the process flow diagram 600, or other operations may be added. Although the charging device 610 and the wearable device 605 are shown performing the operations of the process flow diagram 600, some aspects of some operations may also be performed by one or more other devices. For example, the wearable device 605 may be a wearable ring device or a wearable wrist-worn device.

At 620, the charging device 610 may detect that the wearable device 605 is mounted to the charging device 610 based on a load detection procedure.

At 625, the wearable device 605 may receive, via inductive charging components, a sequence of incrementally decreasing power output levels from the charging device 610, where the power output levels correspond to detected voltage levels at the wearable device 605. The charging device 610 may output, via inductive charging components and a charging-based communication circuitry, a sequence of incrementally decreasing output power levels to the wearable device 605.

Outputting the power levels may include outputting a first power level, and outputting a second power level that is less than the first power level if the link establishment message is not received before an expiration of a configured timer. The first power level may be a starting power level. For example, the charging device 610 may output a starting power level of the sequence of incrementally decreasing output power levels based on detecting the wearable device 605.

At 630, the wearable device 605 may activate charging-based communication circuitry within the wearable device 605 upon detecting a first power output level of the sequence of incrementally decreasing power output levels that falls below an overload protection threshold. The overload protection threshold may be based on a charge level of a plurality of charge levels of the wearable device.

At 635, the wearable device 605 may output, to the charging device 610 and via the inductive charging components and the charging-based communication circuitry, a first message, such as a link establishment message or other type of message. The charging device 610 may receive the link establishment message via the inductive charging components and the charging-based communication circuitry. At the charging device 610, receiving the link establish message may include detecting a modulation in power draw from the wearable device with respect to a baseline charging voltage and converting, via the charging-based communication circuitry, the modulation in the power draw to a series of bits.

At the wearable device 605, outputting the link establishment message may include modulating a power draw with respect to a baseline charging voltage. Upon outputting the link establishment message, the wearable device 605 may activate a reception timer.

In some examples, the wearable device 605 may select a maximum voltage load based on the detected first output power level being greater than the overload protection threshold, and output the link establishment message using the maximum voltage load.

At 640, the wearable device 605 may receive, from the charging device and via the inductive charging components and the charging-based communication circuitry, a second message, such as a link confirmation message or other message in response to the first message (e.g., a link establishment message). The charging device 610 may output the link confirmation message via the inductive charging components and the charging-based communication circuitry. Outputting the link confirmation messages may include modulating an output power with respect to a baseline charging voltage.

At the wearable device 605, receiving the link confirmation message may include detecting a modulation of output power from the charging device, and converting, via the charging-based communication circuitry, the modulation in output power to a series of bits. The wearable device 605 may receive the link confirmation message before expiration of the reception timer.

At 645, the wearable device 605 may output, after outputting the link establishment message to the charging device 610 and via the inductive charging components and the charging-based communication circuitry, one or more additional messages based at least in part on modulations in a power draw with respect to a baseline charging voltage. The wearable device 605 may also receive, after receiving the link confirmation message from the charging device and via the inductive charging components and the charging-based communication circuitry, one or more additional messages based at least in part on modulations in output power with respect to a baseline charging voltage.

Similarly, the charging device 610 may receive and output one or more additional messages based at least in part on modulations in output power with respect to a baseline charging voltage.

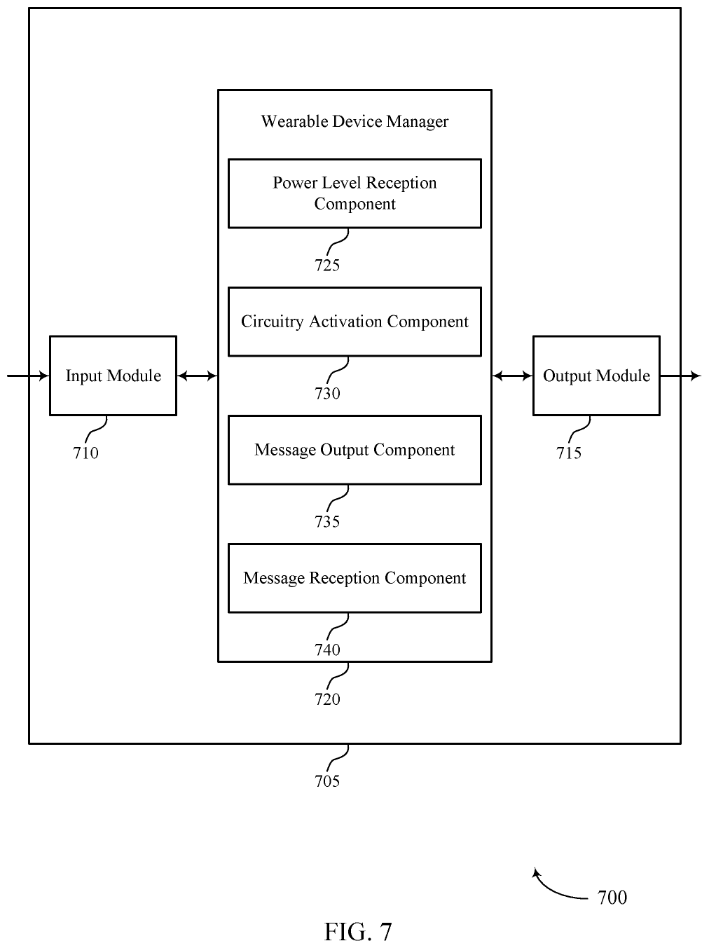

FIG. 7 shows a block diagram 700 of a device 705 that supports charging interface communication in accordance with aspects of the present disclosure. The device 705 may include an input module 710, an output module 715, and a wearable device manager 720. The device 705, or one or more components of the device 705 (e.g., the input module 710, the output module 715, and the wearable device manager 720), may include at least one processor, which may be coupled with at least one memory, to support the described techniques. Each of these components may be in communication with one another (e.g., via one or more buses).

For example, the wearable device manager 720 may include a power level reception component 725, a circuitry activation component 730, a message output component 735, a message reception component 740, or any combination thereof. In some examples, the wearable device manager 720, or various components thereof, may be configured to perform various operations (e.g., receiving, monitoring, transmitting) using or otherwise in cooperation with the input module 710, the output module 715, or both. For example, the wearable device manager 720 may receive information from the input module 710, send information to the output module 715, or be integrated in combination with the input module 710, the output module 715, or both to receive information, transmit information, or perform various other operations as described herein.

The power level reception component 725 may be configured as or otherwise support a means for receiving, via inductive charging components, a sequence of incrementally decreasing power output levels from a charging device, wherein the power output levels correspond to detected voltage levels at the wearable device. The circuitry activation component 730 may be configured as or otherwise support a means for activating charging-based communication circuitry within the wearable device upon detecting a first power output level of the sequence of incrementally decreasing power output levels that falls below an overload protection threshold. The message output component 735 may be configured as or otherwise support a means for outputting, to the charging device and via the inductive charging components and the charging-based communication circuitry, a link establishment message. The message reception component 740 may be configured as or otherwise support a means for receiving, from the charging device and via the inductive charging components and the charging-based communication circuitry, a link confirmation message in response to the link establishment message.

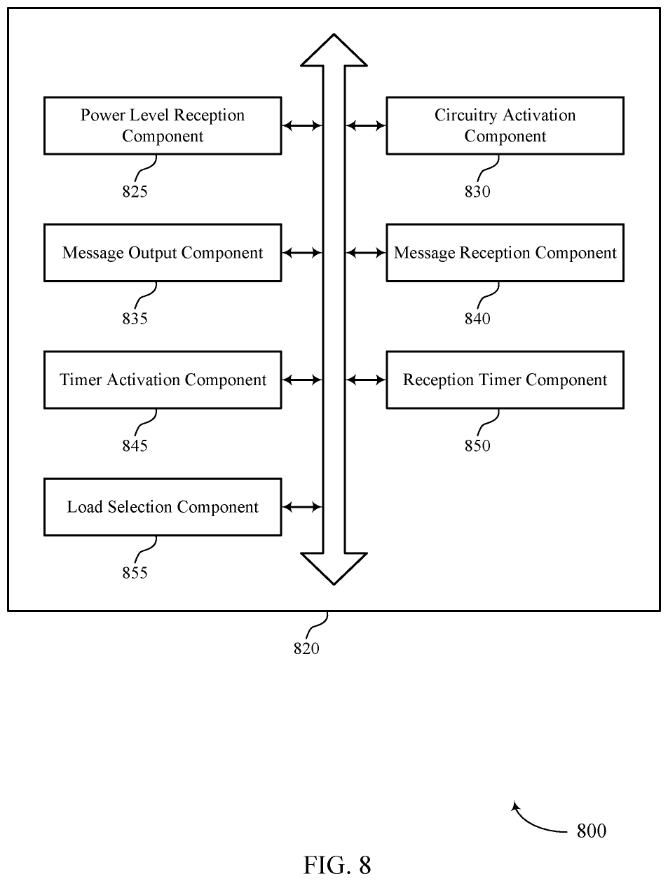

FIG. 8 shows a block diagram 800 of a wearable device manager 820 that supports charging interface communication in accordance with aspects of the present disclosure. The wearable device manager 820 may be an example of aspects of a wearable device manager or a wearable device manager 720, or both, as described herein. The wearable device manager 820, or various components thereof, may be an example of means for performing various aspects of charging interface communication as described herein. For example, the wearable device manager 820 may include a power level reception component 825, a circuitry activation component 830, a message output component 835, a message reception component 840, a timer activation component 845, a reception timer component 850, a load selection component 855, or any combination thereof. Each of these components, or components of subcomponents thereof (e.g., one or more processors, one or more memories), may communicate, directly or indirectly, with one another (e.g., via one or more buses).

The power level reception component 825 may be configured as or otherwise support a means for receiving, via inductive charging components, a sequence of incrementally decreasing power output levels from a charging device, wherein the power output levels correspond to detected voltage levels at the wearable device. The circuitry activation component 830 may be configured as or otherwise support a means for activating charging-based communication circuitry within the wearable device upon detecting a first power output level of the sequence of incrementally decreasing power output levels that falls below an overload protection threshold. The message output component 835 may be configured as or otherwise support a means for outputting, to the charging device and via the inductive charging components and the charging-based communication circuitry, a link establishment message. The message reception component 840 may be configured as or otherwise support a means for receiving, from the charging device and via the inductive charging components and the charging-based communication circuitry, a link confirmation message in response to the link establishment message.

In some examples, to support outputting the link establishment message, the message output component 835 may be configured as or otherwise support a means for modulating a power draw with respect to a baseline charging voltage.

In some examples, to support receiving the link confirmation message in response to the link establishment message, the message reception component 840 may be configured as or otherwise support a means for detecting a modulation of output power from the charging device. In some examples, to support receiving the link confirmation message in response to the link establishment message, the message reception component 840 may be configured as or otherwise support a means for converting, via the charging-based communication circuitry, the modulation in output power to a series of bits.

In some examples, the overload protection threshold is based at least in part on a charge level of a plurality of charge levels of the wearable device.

In some examples, the timer activation component 845 may be configured as or otherwise support a means for activating a reception timer based at least in part on outputting the link establishment message. In some examples, the reception timer component 850 may be configured as or otherwise support a means for receiving the link confirmation message before expiration of the reception timer.

In some examples, the message output component 835 may be configured as or otherwise support a means for outputting, after outputting the link establishment message to the charging device and via the inductive charging components and the charging-based communication circuitry, one or more additional messages based at least in part on modulations in a power draw with respect to a baseline charging voltage.

In some examples, the message reception component 840 may be configured as or otherwise support a means for receiving, after receiving the link confirmation message from the charging device and via the inductive charging components and the charging-based communication circuitry, one or more additional messages based at least in part on modulations in output power with respect to a baseline charging voltage.

In some examples, the load selection component 855 may be configured as or otherwise support a means for selecting a maximum voltage load based at least in part on the detected first output power level being greater than the overload protection threshold. In some examples, the message output component 835 may be configured as or otherwise support a means for outputting the link establishment message using the maximum voltage load.

In some examples, the wearable device is a wearable ring device or a wearable wrist device.

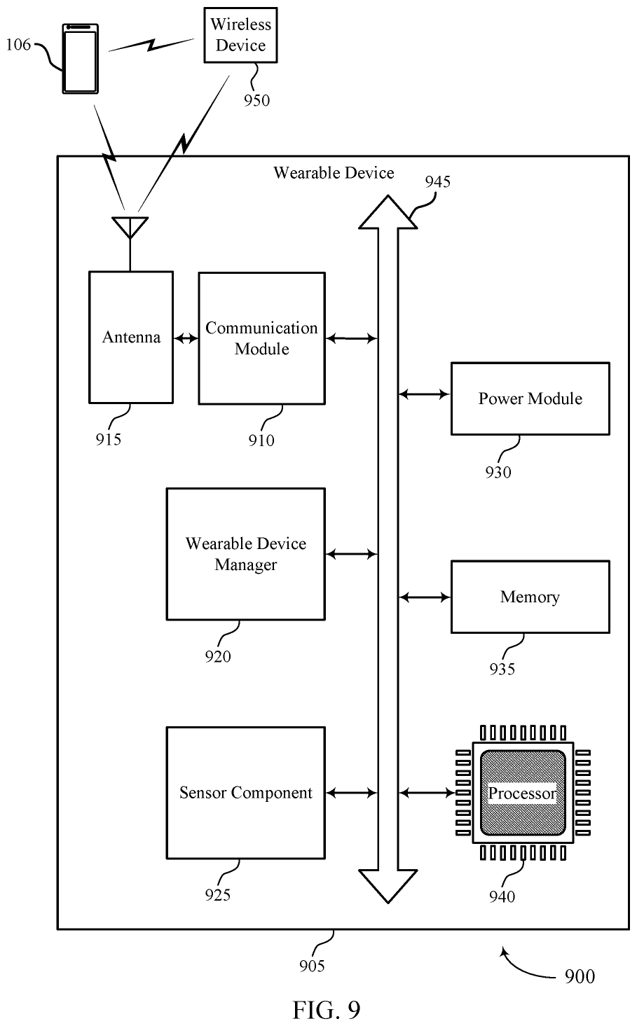

FIG. 9 shows a diagram of a system 900 including a device 905 (e.g., wearable device) that supports charging interface communication in accordance with aspects of the present disclosure. The device 905 may be an example of or include the components of a device 705 as described herein. The device 905 may include an example of a wearable device 104, as described previously herein. The device 905 may include components for bi-directional communications including components for transmitting and receiving communications with a user device 106 and a server 110, such as a wearable device manager 920, a communication module 910, an antenna 915, a sensor component 925, a power module 930, at least one memory 935, at least one processor 940, and a wireless device 950. These components may be in electronic communication or otherwise coupled (e.g., operatively, communicatively, functionally, electronically, electrically) via one or more buses (e.g., a bus 945).

For example, the wearable device manager 920 may be configured as or otherwise support a means for receiving, via inductive charging components, a sequence of incrementally decreasing power output levels from a charging device, wherein the power output levels correspond to detected voltage levels at the wearable device. The wearable device manager 920 may be configured as or otherwise support a means for activating charging-based communication circuitry within the wearable device upon detecting a first power output level of the sequence of incrementally decreasing power output levels that falls below an overload protection threshold. The wearable device manager 920 may be configured as or otherwise support a means for outputting, to the charging device and via the inductive charging components and the charging-based communication circuitry, a link establishment message. The wearable device manager 920 may be configured as or otherwise support a means for receiving, from the charging device and via the inductive charging components and the charging-based communication circuitry, a link confirmation message in response to the link establishment message.

By including or configuring the wearable device manager 920 in accordance with examples as described herein, the device 905 may support techniques for charging interface communication, which may result in various advantages, including but limited to: improved communication reliability, reduced latency, improved user experience related to reduced processing, reduced power consumption, more efficient utilization of communication resources, improved coordination between devices, longer battery life, and improved utilization of processing capability.

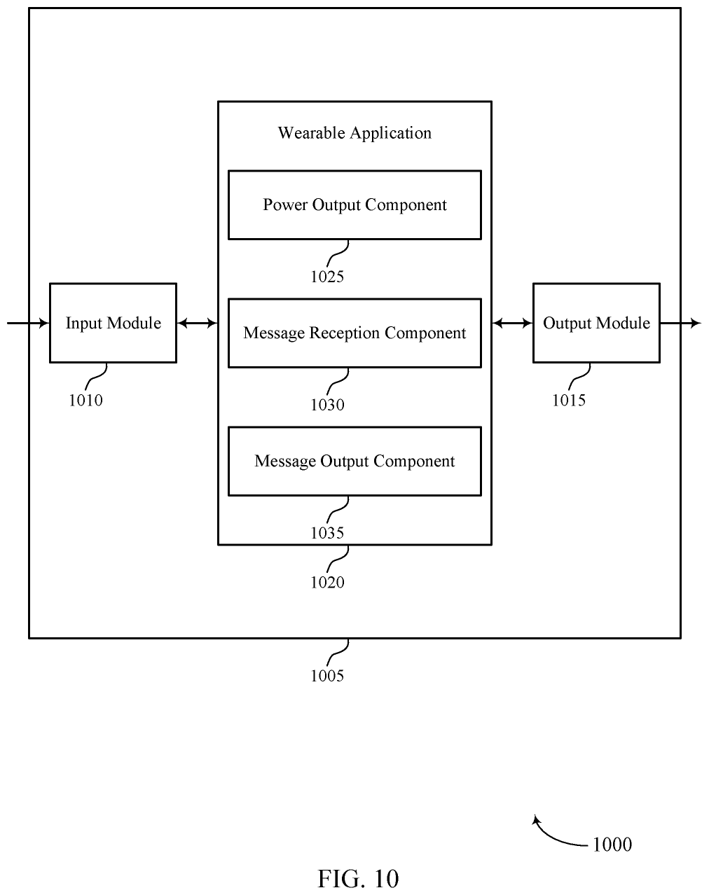

FIG. 10 shows a block diagram 1000 of a device 1005 (e.g., wearable device, charging device) that supports charging interface communication in accordance with aspects of the present disclosure. The device 1005 may include an input module 1010, an output module 1015, and a wearable application 1020. The device 1005, or one or more components of the device 1005 (e.g., the input module 1010, the output module 1015, and the wearable application 1020), may include at least one processor, which may be coupled with at least one memory, to support the described techniques. Each of these components may be in communication with one another (e.g., via one or more buses).

The input module 1010 may provide a means for receiving information such as packets, user data, control information, or any combination thereof associated with various information channels (e.g., control channels, data channels, information channels related to illness detection techniques). Information may be passed on to other components of the device 1005. The input module 1010 may utilize a single antenna or a set of multiple antennas.

The output module 1015 may provide a means for transmitting signals generated by other components of the device 1005. For example, the output module 1015 may transmit information such as packets, user data, control information, or any combination thereof associated with various information channels (e.g., control channels, data channels, information channels related to illness detection techniques). In some examples, the output module 1015 may be co-located with the input module 1010 in a transceiver module. The output module 1015 may utilize a single antenna or a set of multiple antennas.

For example, the wearable application 1020 may include a power output component 1025, a message reception component 1030, a message output component 1035, or any combination thereof. In some examples, the wearable application 1020, or various components thereof, may be configured to perform various operations (e.g., receiving, monitoring, transmitting) using or otherwise in cooperation with the input module 1010, the output module 1015, or both. For example, the wearable application 1020 may receive information from the input module 1010, send information to the output module 1015, or be integrated in combination with the input module 1010, the output module 1015, or both to receive information, transmit information, or perform various other operations as described herein.

The power output component 1025 may be configured as or otherwise support a means for outputting, via inductive charging components and a charging-based communication circuitry, a sequence of incrementally decreasing output power levels to a wearable device, wherein the output power levels correspond to detected voltage levels at the wearable device. The message reception component 1030 may be configured as or otherwise support a means for receiving, from the wearable device and via the inductive charging components and the charging-based communication circuitry, a link establishment message. The message output component 1035 may be configured as or otherwise support a means for outputting, to the wearable device and via the inductive charging components and the charging-based communication circuitry, a link confirmation message in response to the link establishment message.

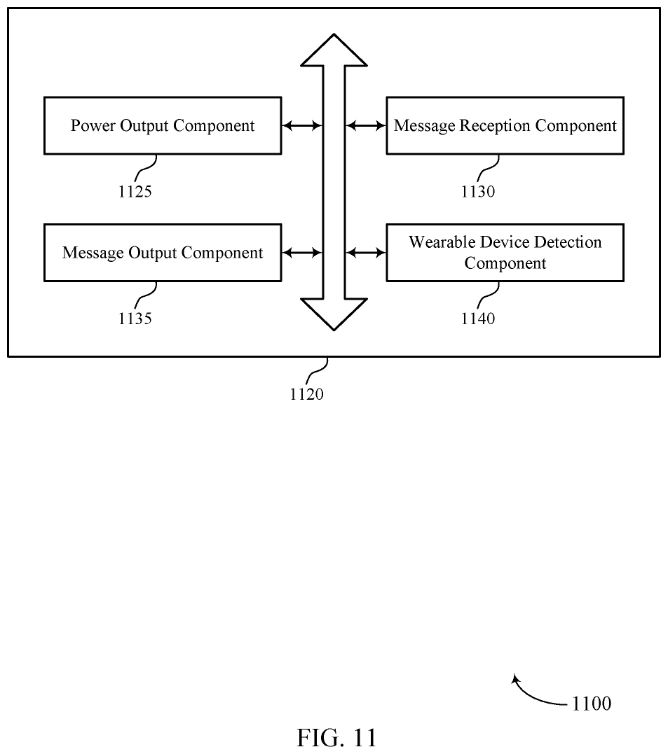

FIG. 11 shows a block diagram 1100 of a wearable application 1120 (e.g., charging device) that supports charging interface communication in accordance with aspects of the present disclosure. The wearable application 1120 may be an example of aspects of a wearable application or a wearable application 1020, or both, as described herein. The wearable application 1120, or various components thereof, may be an example of means for performing various aspects of charging interface communication as described herein. For example, the wearable application 1120 may include a power output component 1125, a message reception component 1130, a message output component 1135, a wearable device detection component 1140, or any combination thereof. Each of these components, or components of subcomponents thereof (e.g., one or more processors, one or more memories), may communicate, directly or indirectly, with one another (e.g., via one or more buses).

The power output component 1125 may be configured as or otherwise support a means for outputting, via inductive charging components and a charging-based communication circuitry, a sequence of incrementally decreasing output power levels to a wearable device, wherein the output power levels correspond to detected voltage levels at the wearable device. The message reception component 1130 may be configured as or otherwise support a means for receiving, from the wearable device and via the inductive charging components and the charging-based communication circuitry, a link establishment message. The message output component 1135 may be configured as or otherwise support a means for outputting, to the wearable device and via the inductive charging components and the charging-based communication circuitry, a link confirmation message in response to the link establishment message.

In some examples, to support outputting the sequence of incrementally decreasing output power levels, the power output component 1125 may be configured as or otherwise support a means for outputting a first power level. In some examples, to support outputting the sequence of incrementally decreasing output power levels, the power output component 1125 may be configured as or otherwise support a means for outputting a second power level that is less than the first power level if the link establishment message is not received before an expiration of a configured timer.

In some examples, the wearable device detection component 1140 may be configured as or otherwise support a means for detecting the wearable device is mounted to the charging device based at least in part on a load detection procedure. In some examples, the power output component 1125 may be configured as or otherwise support a means for outputting a starting power level of the sequence of incrementally decreasing output power levels based at least in part on detecting the wearable device.

In some examples, to support receiving the link establishment message, the message reception component 1130 may be configured as or otherwise support a means for detecting a modulation in power draw from the wearable device with respect to a baseline charging voltage. In some examples, to support receiving the link establishment message, the message reception component 1130 may be configured as or otherwise support a means for converting, via the charging-based communication circuitry, the modulation in the power draw to a series of bits.

In some examples, to support outputting the link confirmation message in response to the link establishment message, the message output component 1135 may be configured as or otherwise support a means for modulating an output power with respect to a baseline charging voltage.

In some examples, the message reception component 1130 may be configured as or otherwise support a means for receiving, after receiving the link establishment message from the wearable device and via the inductive charging components and the charging-based communication circuitry, one or more additional messages based at least in part on modulations in power draw with respect to a baseline charging voltage.

In some examples, the message output component 1135 may be configured as or otherwise support a means for outputting, after outputting the link confirmation message to the wearable device and via the inductive charging components and the charging-based communication circuitry, one or more additional messages based at least in part on modulations in output power with respect to a baseline charging voltage.

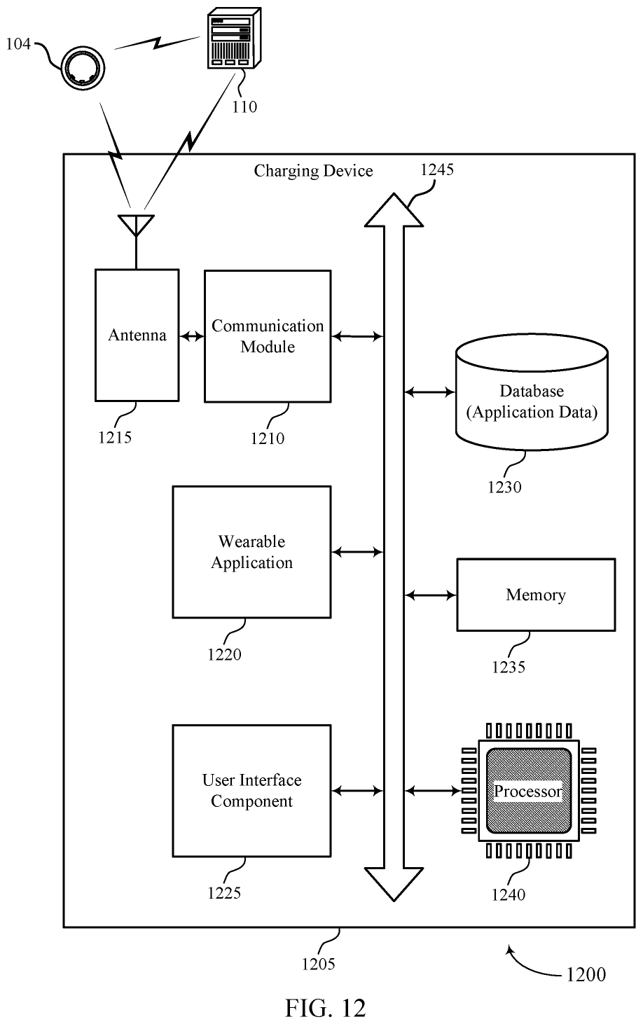

FIG. 12 shows a diagram of a system 1200 including a charging device 1205 that supports charging interface communication in accordance with aspects of the present disclosure. The charging device 1205 may be an example of or include the components of a device 1005 as described herein. The charging device 1205 may include an example of a user device 106, as described previously herein. The charging device 1205 may include components for bi-directional communications including components for transmitting and receiving communications with a wearable device 104 and a server 110, such as a wearable application 1220, a communication module 1210, an antenna 1215, a user interface component 1225, a database (application data) 1230, at least one memory 1235, and at least one processor 1240. These components may be in electronic communication or otherwise coupled (e.g., operatively, communicatively, functionally, electronically, electrically) via one or more buses (e.g., a bus 1245).

The communication module 1210 may manage input and output signals for the charging device 1205 via the antenna 1215. The communication module 1210 may include an example of the communication module 220-b of the user device 106 shown and described in FIG. 2. In this regard, the communication module 1210 may manage communications with the ring 104 and the server 110, as illustrated in FIG. 2. The communication module 1210 may also manage peripherals not integrated into the charging device 1205. In some cases, the communication module 1210 may represent a physical connection or port to an external peripheral. In some cases, the communication module 1210 may utilize an operating system such as iOS®, ANDROID®, MS-DOS®, MS-WINDOWS®, OS/2®, UNIX®, LINUX®, or another known operating system. In other cases, the communication module 1210 may represent or interact with a wearable device (e.g., ring 104), modem, a keyboard, a mouse, a touchscreen, or a similar device. In some cases, the communication module 1210 may be implemented as part of the processor 1240. In some examples, a user may interact with the charging device 1205 via the communication module 1210, user interface component 1225, or via hardware components controlled by the communication module 1210.

In some cases, the charging device 1205 may include a single antenna 1215. However, in some other cases, the charging device 1205 may have more than one antenna 1215, which may be capable of concurrently transmitting or receiving multiple wireless transmissions. The communication module 1210 may communicate bi-directionally, via the one or more antennas 1215, wired, or wireless links as described herein. For example, the communication module 1210 may represent a wireless transceiver and may communicate bi-directionally with another wireless transceiver. The communication module 1210 may also include a modem to modulate the packets, to provide the modulated packets to one or more antennas 1215 for transmission, and to demodulate packets received from the one or more antennas 1215.

The user interface component 1225 may manage data storage and processing in a database 1230. In some cases, a user may interact with the user interface component 1225. In other cases, the user interface component 1225 may operate automatically without user interaction. The database 1230 may be an example of a single database, a distributed database, multiple distributed databases, a data store, a data lake, or an emergency backup database.

The memory 1235 may include RAM and ROM. The memory 1235 may store computer-readable, computer-executable software including instructions that, when executed, cause the processor 1240 to perform various functions described herein. In some cases, the memory 1235 may contain, among other things, a BIOS which may control basic hardware or software operation such as the interaction with peripheral components or devices.

The processor 1240 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, the processor 1240 may be configured to operate a memory array using a memory controller. In other cases, a memory controller may be integrated into the processor 1240. The processor 1240 may be configured to execute computer-readable instructions stored in a memory 1235 to perform various functions (e.g., functions or tasks supporting a method and system for sleep staging algorithms).

For example, the wearable application 1220 may be configured as or otherwise support a means for outputting, via inductive charging components and a charging-based communication circuitry, a sequence of incrementally decreasing output power levels to a wearable device, wherein the output power levels correspond to detected voltage levels at the wearable device. The wearable application 1220 may be configured as or otherwise support a means for receiving, from the wearable device and via the inductive charging components and the charging-based communication circuitry, a link establishment message. The wearable application 1220 may be configured as or otherwise support a means for outputting, to the wearable device and via the inductive charging components and the charging-based communication circuitry, a link confirmation message in response to the link establishment message.

By including or configuring the wearable application 1220 in accordance with examples as described herein, the charging device 1205 may support techniques for charging interface communication, which may result in various advantages, including but limited to: improved communication reliability, reduced latency, improved user experience related to reduced processing, reduced power consumption, more efficient utilization of communication resources, improved coordination between devices, longer battery life, and improved utilization of processing capability.

The wearable application 1220 may include an application (e.g., “app”), program, software, or other component which is configured to facilitate communications with a ring 104, server 110, other user devices 106, and the like. For example, the wearable application 1220 may include an application executable on a user device 106 which is configured to receive data (e.g., physiological data) from a ring 104, perform processing operations on the received data, transmit and receive data with the servers 110, and cause presentation of data to a user 102.

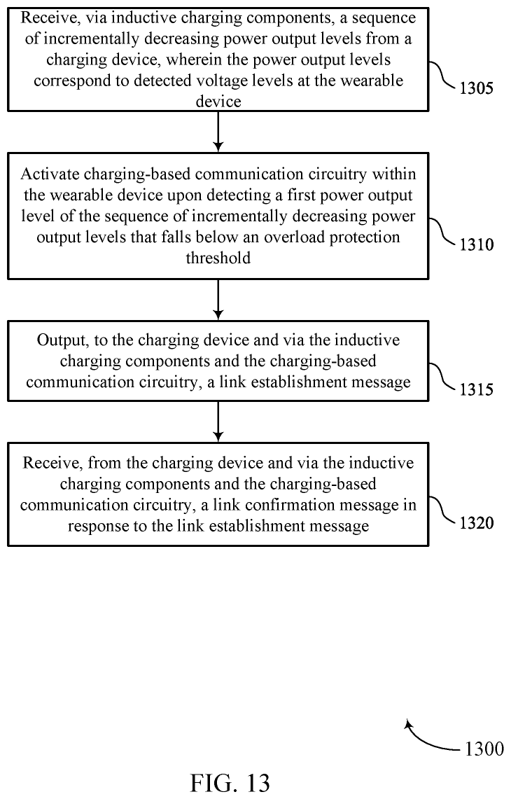

FIG. 13 shows a flowchart illustrating a method 1300 that supports charging interface communication in accordance with aspects of the present disclosure. The operations of the method 1300 may be implemented by a wearable device or its components as described herein. For example, the operations of the method 1300 may be performed by a wearable device as described with reference to FIGS. 1 through 9. In some examples, a wearable device may execute a set of instructions to control the functional elements of the wearable device to perform the described functions. Additionally, or alternatively, the wearable device may perform aspects of the described functions using special-purpose hardware.

At 1305, the method may include receiving, via inductive charging components, a sequence of incrementally decreasing power output levels from a charging device, wherein the power output levels correspond to detected voltage levels at the wearable device. The operations of block 1305 may be performed in accordance with examples as disclosed herein. In some examples, aspects of the operations of 1305 may be performed by a power level reception component 825 as described with reference to FIG. 8.

At 1310, the method may include activating charging-based communication circuitry within the wearable device upon detecting a first power output level of the sequence of incrementally decreasing power output levels that falls below an overload protection threshold. The operations of block 1310 may be performed in accordance with examples as disclosed herein. In some examples, aspects of the operations of 1310 may be performed by a circuitry activation component 830 as described with reference to FIG. 8.

At 1315, the method may include outputting, to the charging device and via the inductive charging components and the charging-based communication circuitry, a link establishment message. The operations of block 1315 may be performed in accordance with examples as disclosed herein. In some examples, aspects of the operations of 1315 may be performed by a message output component 835 as described with reference to FIG. 8.

At 1320, the method may include receiving, from the charging device and via the inductive charging components and the charging-based communication circuitry, a link confirmation message in response to the link establishment message. The operations of block 1320 may be performed in accordance with examples as disclosed herein. In some examples, aspects of the operations of 1320 may be performed by a message reception component 840 as described with reference to FIG. 8.

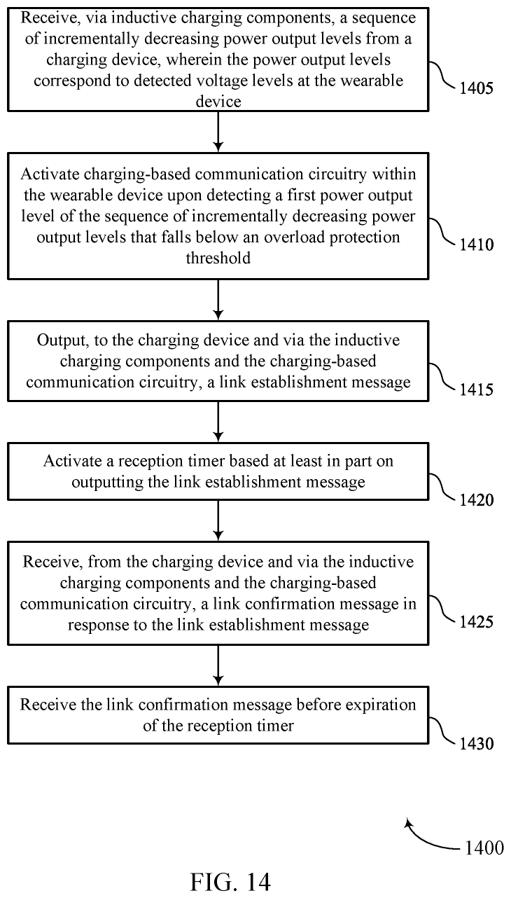

FIG. 14 shows a flowchart illustrating a method 1400 that supports charging interface communication in accordance with aspects of the present disclosure. The operations of the method 1400 may be implemented by a wearable device or its components as described herein. For example, the operations of the method 1400 may be performed by a wearable device as described with reference to FIGS. 1 through 9. In some examples, a wearable device may execute a set of instructions to control the functional elements of the wearable device to perform the described functions. Additionally, or alternatively, the wearable device may perform aspects of the described functions using special-purpose hardware.

At 1405, the method may include receiving, via inductive charging components, a sequence of incrementally decreasing power output levels from a charging device, wherein the power output levels correspond to detected voltage levels at the wearable device. The operations of block 1405 may be performed in accordance with examples as disclosed herein. In some examples, aspects of the operations of 1405 may be performed by a power level reception component 825 as described with reference to FIG. 8.

At 1410, the method may include activating charging-based communication circuitry within the wearable device upon detecting a first power output level of the sequence of incrementally decreasing power output levels that falls below an overload protection threshold. The operations of block 1410 may be performed in accordance with examples as disclosed herein. In some examples, aspects of the operations of 1410 may be performed by a circuitry activation component 830 as described with reference to FIG. 8.

At 1415, the method may include outputting, to the charging device and via the inductive charging components and the charging-based communication circuitry, a link establishment message. The operations of block 1415 may be performed in accordance with examples as disclosed herein. In some examples, aspects of the operations of 1415 may be performed by a message output component 835 as described with reference to FIG. 8.

At 1420, the method may include activating a reception timer based at least in part on outputting the link establishment message. The operations of block 1420 may be performed in accordance with examples as disclosed herein. In some examples, aspects of the operations of 1420 may be performed by a timer activation component 845 as described with reference to FIG. 8.

At 1425, the method may include receiving, from the charging device and via the inductive charging components and the charging-based communication circuitry, a link confirmation message in response to the link establishment message. The operations of block 1425 may be performed in accordance with examples as disclosed herein. In some examples, aspects of the operations of 1425 may be performed by a message reception component 840 as described with reference to FIG. 8.

At 1430, the method may include receiving the link confirmation message before expiration of the reception timer. The operations of block 1430 may be performed in accordance with examples as disclosed herein. In some examples, aspects of the operations of 1430 may be performed by a reception timer component 850 as described with reference to FIG. 8.

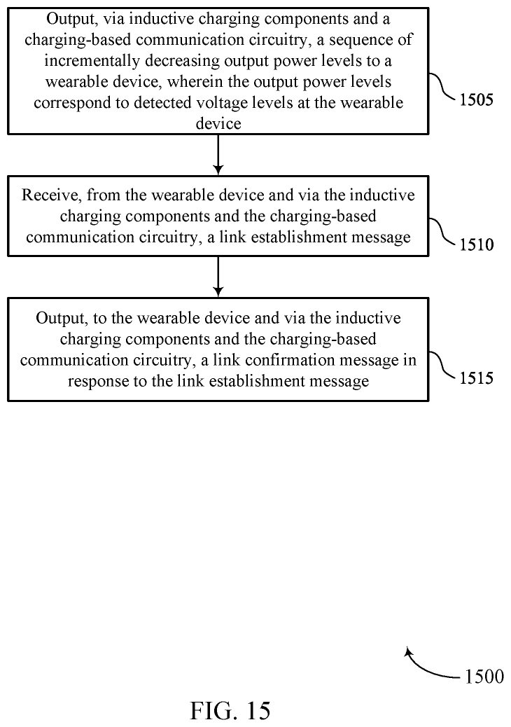

FIG. 15 shows a flowchart illustrating a method 1500 that supports charging interface communication in accordance with aspects of the present disclosure. The operations of the method 1500 may be implemented by a charging device or its components as described herein. For example, the operations of the method 1500 may be performed by a charging device as described with reference to FIGS. 1 through 6 and 10 through 12. In some examples, a charging device may execute a set of instructions to control the functional elements of the charging device to perform the described functions. Additionally, or alternatively, the user device may perform aspects of the described functions using special-purpose hardware.

At 1505, the method may include outputting, via inductive charging components and a charging-based communication circuitry, a sequence of incrementally decreasing output power levels to a wearable device, wherein the output power levels correspond to detected voltage levels at the wearable device. The operations of block 1505 may be performed in accordance with examples as disclosed herein. In some examples, aspects of the operations of 1505 may be performed by a power output component 1125 as described with reference to FIG. 11.

At 1510, the method may include receiving, from the wearable device and via the inductive charging components and the charging-based communication circuitry, a link establishment message. The operations of block 1510 may be performed in accordance with examples as disclosed herein. In some examples, aspects of the operations of 1510 may be performed by a message reception component 1130 as described with reference to FIG. 11.

At 1515, the method may include outputting, to the wearable device and via the inductive charging components and the charging-based communication circuitry, a link confirmation message in response to the link establishment message. The operations of block 1515 may be performed in accordance with examples as disclosed herein. In some examples, aspects of the operations of 1515 may be performed by a message output component 1135 as described with reference to FIG. 11.

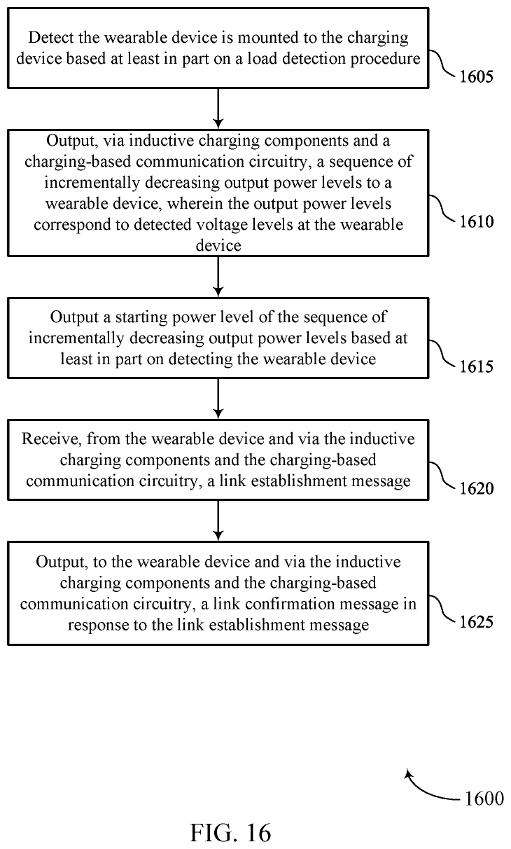

FIG. 16 shows a flowchart illustrating a method 1600 that supports charging interface communication in accordance with aspects of the present disclosure. The operations of the method 1600 may be implemented by a charging device or its components as described herein. For example, the operations of the method 1600 may be performed by a charging device as described with reference to FIGS. 1 through 6 and 10 through 12. In some examples, a charging device may execute a set of instructions to control the functional elements of the user device to perform the described functions. Additionally, or alternatively, the charging device may perform aspects of the described functions using special-purpose hardware.

At 1605, the method may include detecting the wearable device is mounted to the charging device based at least in part on a load detection procedure. The operations of block 1605 may be performed in accordance with examples as disclosed herein. In some examples, aspects of the operations of 1605 may be performed by a wearable device detection component 1140 as described with reference to FIG. 11.

At 1610, the method may include outputting, via inductive charging components and a charging-based communication circuitry, a sequence of incrementally decreasing output power levels to a wearable device, wherein the output power levels correspond to detected voltage levels at the wearable device. The operations of block 1610 may be performed in accordance with examples as disclosed herein. In some examples, aspects of the operations of 1610 may be performed by a power output component 1125 as described with reference to FIG. 11.

At 1615, the method may include outputting a starting power level of the sequence of incrementally decreasing output power levels based at least in part on detecting the wearable device. The operations of block 1615 may be performed in accordance with examples as disclosed herein. In some examples, aspects of the operations of 1615 may be performed by a power output component 1125 as described with reference to FIG. 11.

At 1620, the method may include receiving, from the wearable device and via the inductive charging components and the charging-based communication circuitry, a link establishment message. The operations of block 1620 may be performed in accordance with examples as disclosed herein. In some examples, aspects of the operations of 1620 may be performed by a message reception component 1130 as described with reference to FIG. 11.

At 1625, the method may include outputting, to the wearable device and via the inductive charging components and the charging-based communication circuitry, a link confirmation message in response to the link establishment message. The operations of block 1625 may be performed in accordance with examples as disclosed herein. In some examples, aspects of the operations of 1625 may be performed by a message output component 1135 as described with reference to FIG. 11.

It should be noted that the methods described above describe possible implementations, and that the operations and the steps may be rearranged or otherwise modified and that other implementations are possible. Furthermore, aspects from two or more of the methods may be combined.

A method by a wearable device is described. The method may include receiving, via inductive charging components, a sequence of incrementally decreasing power output levels from a charging device, wherein the power output levels correspond to detected voltage levels at the wearable device, activating charging-based communication circuitry within the wearable device upon detecting a first power output level of the sequence of incrementally decreasing power output levels that falls below an overload protection threshold, outputting, to the charging device and via the inductive charging components and the charging-based communication circuitry, a link establishment message, and receiving, from the charging device and via the inductive charging components and the charging-based communication circuitry, a link confirmation message in response to the link establishment message.

A wearable device is described. The wearable device may include one or more memories storing processor executable code, and one or more processors coupled with the one or more memories. The one or more processors may individually or collectively operable to execute the code to cause the wearable device to receive, via inductive charging components, a sequence of incrementally decreasing power output levels from a charging device, wherein the power output levels correspond to detected voltage levels at the wearable device, activate charging-based communication circuitry within the wearable device upon detecting a first power output level of the sequence of incrementally decreasing power output levels that falls below an overload protection threshold, output, to the charging device and via the inductive charging components and the charging-based communication circuitry, a link establishment message, and receive, from the charging device and via the inductive charging components and the charging-based communication circuitry, a link confirmation message in response to the link establishment message.

Another wearable device is described. The wearable device may include means for receiving, via inductive charging components, a sequence of incrementally decreasing power output levels from a charging device, wherein the power output levels correspond to detected voltage levels at the wearable device, means for activating charging-based communication circuitry within the wearable device upon detecting a first power output level of the sequence of incrementally decreasing power output levels that falls below an overload protection threshold, means for outputting, to the charging device and via the inductive charging components and the charging-based communication circuitry, a link establishment message, and means for receiving, from the charging device and via the inductive charging components and the charging-based communication circuitry, a link confirmation message in response to the link establishment message.

A non-transitory computer-readable medium storing code is described. The code may include instructions executable by one or more processors to receive, via inductive charging components, a sequence of incrementally decreasing power output levels from a charging device, wherein the power output levels correspond to detected voltage levels at the wearable device, activate charging-based communication circuitry within the wearable device upon detecting a first power output level of the sequence of incrementally decreasing power output levels that falls below an overload protection threshold, output, to the charging device and via the inductive charging components and the charging-based communication circuitry, a link establishment message, and receive, from the charging device and via the inductive charging components and the charging-based communication circuitry, a link confirmation message in response to the link establishment message.

In some examples of the method, wearable devices, and non-transitory computer-readable medium described herein, outputting the link establishment message may include operations, features, means, or instructions for modulating a power draw with respect to a baseline charging voltage.

In some examples of the method, wearable devices, and non-transitory computer-readable medium described herein, receiving the link confirmation message in response to the link establishment message may include operations, features, means, or instructions for detecting a modulation of output power from the charging device and converting, via the charging-based communication circuitry, the modulation in output power to a series of bits.

In some examples of the method, wearable devices, and non-transitory computer-readable medium described herein, the overload protection threshold may be based at least in part on a charge level of a plurality of charge levels of the wearable device.

Some examples of the method, wearable devices, and non-transitory computer-readable medium described herein may further include operations, features, means, or instructions for activating a reception timer based at least in part on outputting the link establishment message and receiving the link confirmation message before expiration of the reception timer.

Some examples of the method, wearable devices, and non-transitory computer-readable medium described herein may further include operations, features, means, or instructions for outputting, after outputting the link establishment message to the charging device and via the inductive charging components and the charging-based communication circuitry, one or more additional messages based at least in part on modulations in a power draw with respect to a baseline charging voltage.

Some examples of the method, wearable devices, and non-transitory computer-readable medium described herein may further include operations, features, means, or instructions for receiving, after receiving the link confirmation message from the charging device and via the inductive charging components and the charging-based communication circuitry, one or more additional messages based at least in part on modulations in output power with respect to a baseline charging voltage.

Some examples of the method, wearable devices, and non-transitory computer-readable medium described herein may further include operations, features, means, or instructions for selecting a maximum voltage load based at least in part on the detected first output power level being greater than the overload protection threshold and outputting the link establishment message using the maximum voltage load.

In some examples of the method, wearable devices, and non-transitory computer-readable medium described herein, the wearable device may be a wearable ring device or a wearable wrist device.

A method by a charging device is described. The method may include outputting, via inductive charging components and a charging-based communication circuitry, a sequence of incrementally decreasing output power levels to a wearable device, wherein the output power levels correspond to detected voltage levels at the wearable device, receiving, from the wearable device and via the inductive charging components and the charging-based communication circuitry, a link establishment message, and outputting, to the wearable device and via the inductive charging components and the charging-based communication circuitry, a link confirmation message in response to the link establishment message.

A charging device is described. The charging device may include one or more memories storing processor executable code, and one or more processors coupled with the one or more memories. The one or more processors may individually or collectively operable to execute the code to cause the charging device to output, via inductive charging components and a charging-based communication circuitry, a sequence of incrementally decreasing output power levels to a wearable device, wherein the output power levels correspond to detected voltage levels at the wearable device, receive, from the wearable device and via the inductive charging components and the charging-based communication circuitry, a link establishment message, and output, to the wearable device and via the inductive charging components and the charging-based communication circuitry, a link confirmation message in response to the link establishment message.

Another charging device is described. The charging device may include means for outputting, via inductive charging components and a charging-based communication circuitry, a sequence of incrementally decreasing output power levels to a wearable device, wherein the output power levels correspond to detected voltage levels at the wearable device, means for receiving, from the wearable device and via the inductive charging components and the charging-based communication circuitry, a link establishment message, and means for outputting, to the wearable device and via the inductive charging components and the charging-based communication circuitry, a link confirmation message in response to the link establishment message.

A non-transitory computer-readable medium storing code is described. The code may include instructions executable by one or more processors to output, via inductive charging components and a charging-based communication circuitry, a sequence of incrementally decreasing output power levels to a wearable device, wherein the output power levels correspond to detected voltage levels at the wearable device, receive, from the wearable device and via the inductive charging components and the charging-based communication circuitry, a link establishment message, and output, to the wearable device and via the inductive charging components and the charging-based communication circuitry, a link confirmation message in response to the link establishment message.

In some examples of the method, charging devices, and non-transitory computer-readable medium described herein, outputting the sequence of incrementally decreasing output power levels may include operations, features, means, or instructions for outputting a first power level and outputting a second power level that may be less than the first power level if the link establishment message may be not received before an expiration of a configured timer.

Some examples of the method, charging devices, and non-transitory computer-readable medium described herein may further include operations, features, means, or instructions for detecting the wearable device may be mounted to the charging device based at least in part on a load detection procedure and outputting a starting power level of the sequence of incrementally decreasing output power levels based at least in part on detecting the wearable device.

In some examples of the method, charging devices, and non-transitory computer-readable medium described herein, receiving the link establishment message may include operations, features, means, or instructions for detecting a modulation in power draw from the wearable device with respect to a baseline charging voltage and converting, via the charging-based communication circuitry, the modulation in the power draw to a series of bits.

In some examples of the method, charging devices, and non-transitory computer-readable medium described herein, outputting the link confirmation message in response to the link establishment message may include operations, features, means, or instructions for modulating an output power with respect to a baseline charging voltage.

Some examples of the method, charging devices, and non-transitory computer-readable medium described herein may further include operations, features, means, or instructions for receiving, after receiving the link establishment message from the wearable device and via the inductive charging components and the charging-based communication circuitry, one or more additional messages based at least in part on modulations in power draw with respect to a baseline charging voltage.

Some examples of the method, charging devices, and non-transitory computer-readable medium described herein may further include operations, features, means, or instructions for outputting, after outputting the link confirmation message to the wearable device and via the inductive charging components and the charging-based communication circuitry, one or more additional messages based at least in part on modulations in output power with respect to a baseline charging voltage.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method at a wearable device, comprising:
receiving, via inductive charging components, a sequence of incrementally decreasing power output levels from a charging device, wherein the power output levels correspond to detected voltage levels at the wearable device;
activating charging-based communication circuitry within the wearable device upon detecting a first power output level of the sequence of incrementally decreasing power output levels that falls below an overload protection threshold;
outputting, to the charging device and via the inductive charging components and the charging-based communication circuitry, a first message; and
receiving, from the charging device and via the inductive charging components and the charging-based communication circuitry, a second message in response to the first message.

2. The method of claim 1, wherein outputting the first message further comprises:
modulating a power draw with respect to a baseline charging voltage.

3. The method of claim 1, wherein receiving the second message in response to the first message further comprises:
detecting a modulation of output power from the charging device; and
converting, via the charging-based communication circuitry, the modulation in output power to a series of bits.

4. The method of claim 1, wherein the overload protection threshold is based at least in part on a charge level of a plurality of charge levels of the wearable device.

5. The method of claim 1, further comprising:
activating a reception timer based at least in part on outputting the first message; and
receiving the second message before expiration of the reception timer.

6. The method of claim 1, further comprising:
outputting, after outputting the first message to the charging device and via the inductive charging components and the charging-based communication circuitry, one or more additional messages based at least in part on modulations in a power draw with respect to a baseline charging voltage.

7. The method of claim 1, further comprising:
receiving, after receiving the second message from the charging device and via the inductive charging components and the charging-based communication circuitry, one or more additional messages based at least in part on modulations in output power with respect to a baseline charging voltage.

8. The method of claim 1, further comprising:
selecting a maximum voltage load based at least in part on the detected first output power level being greater than the overload protection threshold; and
outputting the first message using the maximum voltage load.

9. The method of claim 1, wherein the wearable device is a wearable ring device or a wearable wrist-worn device.

10. The method of claim 1, wherein the first message comprises a link establishment message and the second message comprises a link confirmation message.

11. A method at a charging device, comprising:
outputting, via inductive charging components and a charging-based communication circuitry, a sequence of incrementally decreasing output power levels to a wearable device, wherein the output power levels correspond to detected voltage levels at the wearable device;
receiving, from the wearable device and via the inductive charging components and the charging-based communication circuitry, a first message; and
outputting, to the wearable device and via the inductive charging components and the charging-based communication circuitry, a second message in response to the first message.

12. The method of claim 11, wherein outputting the sequence of incrementally decreasing output power levels comprises:
outputting a first power level; and
outputting a second power level that is less than the first power level if the first message is not received before an expiration of a configured timer.

13. The method of claim 11, further comprising:
detecting the wearable device is mounted to the charging device based at least in part on a load detection procedure; and
outputting a starting power level of the sequence of incrementally decreasing output power levels based at least in part on detecting the wearable device.

14. The method of claim 11, wherein receiving the first message further comprises:
detecting a modulation in power draw from the wearable device with respect to a baseline charging voltage; and
converting, via the charging-based communication circuitry, the modulation in the power draw to a series of bits.

15. The method of claim 11, wherein outputting the second message in response to the first message comprises:
modulating an output power with respect to a baseline charging voltage.

16. The method of claim 11, further comprising:
receiving, after receiving the first message from the wearable device and via the inductive charging components and the charging-based communication circuitry, one or more additional messages based at least in part on modulations in power draw with respect to a baseline charging voltage.

17. The method of claim 11, further comprising:
outputting, after outputting the second message to the wearable device and via the inductive charging components and the charging-based communication circuitry, one or more additional messages based at least in part on modulations in output power with respect to a baseline charging voltage.

18. A wearable device, comprising:
one or more memories storing processor-executable code; and
one or more processors coupled with the one or more memories and individually or collectively operable to execute the code to cause the wearable device to:
receive, via inductive charging components, a sequence of incrementally decreasing power output levels from a charging device, wherein the power output levels correspond to detected voltage levels at the wearable device;
activate charging-based communication circuitry within the wearable device upon detecting a first power output level of the sequence of incrementally decreasing power output levels that falls below an overload protection threshold;
output, to the charging device and via the inductive charging components and the charging-based communication circuitry, a first message; and
receive, from the charging device and via the inductive charging components and the charging-based communication circuitry, a second message in response to the first message.

19. The wearable device of claim 18, wherein, to output the first message, the one or more processors are individually or collectively further operable to execute the code to cause the wearable device to:
modulate a power draw with respect to a baseline charging voltage.

20. The wearable device of claim 18, wherein, to receive the second message in response to the first message, the one or more processors are individually or collectively further operable to execute the code to cause the wearable device to:
detect a modulation of output power from the charging device; and
convert, via the charging-based communication circuitry, the modulation in output power to a series of bits.
